(19) United States
(12) Patent Application Publication
(10) Pub. No.: US 2025/0241604 A1
(43) Pub. Date: Jul. 31, 2025

(54) HEAD IMAGE ACQUISITION DEVICE AND HEAD IMAGE ACQUISITION METHOD

(71) Applicant: HAMAMATSU PHOTONICS K.K., Hamamatsu-shi, Shizuoka (JP)

(72) Inventors: Hidemoto YAMAUCHI, Hamamatsu-shi, Shizuoka (JP); Nakahiro SATOH, Hamamatsu-shi, Shizuoka (JP); Kento HAKAMATA, Hamamatsu-shi, Shizuoka (JP)

(73) Assignee: HAMAMATSU PHOTONICS K.K., Hamamatsu-shi, Shizuoka (JP)

(21) Appl. No.: 18/855,069

(22) PCT Filed: Apr. 11, 2023
(86) PCT No.: PCT/JP2023/014712

§ 371 Date: Oct. 8, 2024

(30) Foreign Application Priority Data
Apr. 12, 2022 (JP) 2022-065795

(51) Int. Cl. A61B 6/04; A61B 6/03

(52) U.S. Cl. CPC A61B 6/0421; A61B 6/035; A61B 6/0478

(57) ABSTRACT

A head image acquisition device is a head image acquisition device for acquiring an image of a head of a subject in a sitting state and includes a chair for the subject to sit on, a base, an information detection unit including a detection unit that detects radiation from the subject, and a headrest to which the head of the subject is fixed. The information detection unit is connected to the base so that the information detection unit is movable relative to the base. The headrest is connected to one of the information detection unit and the base so that the headrest is movable relative to the one of the information detection unit and the base.

### TECHNICAL FIELD

One aspect of the present disclosure relates to a head image acquisition device and a head image acquisition method.

### BACKGROUND ART

Patent Literature 1 describes a CT (Computed Tomography) device including a support body and a gantry (information detection unit) provided to be vertically movable and rotatable relative to the support body. In this CT device, a chair including a headrest is provided on the support body. When using this CT device, a head of a subject sitting on the chair is fixed to the headrest. Then, by moving the chair relative to the support body, a position of the gantry relative to the head is adjusted. This alignment is performed using a floodlight installed on the gantry.

### CITATION LIST

### Patent Literature

Patent Literature 1: Japanese Examined Patent Publication No. H8-4586

### SUMMARY OF INVENTION

### Technical Problem

In the above-mentioned CT device, there is a possibility that the chair including the headrest will come into contact with the gantry when aligning the head of the subject. Furthermore, the above-mentioned CT device is required to facilitate alignment between the head of the subject (headrest) and the gantry.

An object of one aspect of the present disclosure provides a head image acquisition device and a head image acquisition method capable of suppressing contact between a headrest and an information detection unit and facilitating alignment between the headrest and the information detection unit.

### Solution to Problem

A head image acquisition device according to an aspect of the present disclosure is [1] “a head image acquisition device for acquiring an image of a head of a subject in a sitting state, the head image acquisition device including a base, an information detection unit including a detection unit that detects radiation from the subject, and a headrest to which the head of the subject is fixed, wherein the information detection unit is connected to the base so that the information detection unit is movable relative to the base, and the headrest is connected to one of the information detection unit and the base so that the headrest is movable relative to the one of the information detection unit and the base”.

In this head image acquisition device, the headrest is connected to the one of the information detection unit and the base so that the headrest is movable relative to the one of the information detection unit and the base. When the one is the base, alignment between the headrest and the information detection unit can be performed by moving the information detection unit relative to the base after fixing the head of the subject to the headrest. Alternatively, when the one is the information detection unit, alignment between the headrest and the information detection unit can be performed by moving the headrest relative to the information detection unit while moving the information detection unit relative to the base after fixing the head of the subject to the headrest. In this head image acquisition device, the headrest is connected to one of the information detection unit and the base. Therefore, for example, when compared to the case where the headrest is provided on the chair, it is possible to suppress occurrence of a situation in which the headrest and the information detection unit are in contact with each other. In addition, since a relative position between the headrest and the information detection unit can be easily structurally determined, it is possible to facilitate alignment between the headrest (the head of the subject) and the information detection unit. Therefore, according to this head image acquisition device, it is possible to suppress contact between the headrest and the information detection unit, and to facilitate alignment between the headrest and the information detection unit.

A head image acquisition device according to an aspect of the

present disclosure may be [2] “the head image acquisition device according to [1], wherein a direction in which the information detection unit moves relative to the base is parallel to a direction in which the headrest moves relative to the one of the information detection unit and the base”. In this case, contact between the headrest and the information detection unit can be further suppressed.

A head image acquisition device according to an aspect of the present disclosure may be [3] “the head image acquisition device according to [1] or [2], further including a first actuator for moving the information detection unit relative to the base, and a second actuator for moving the headrest relative to the one of the information detection unit and the base”. In this case, the information detection unit and the headrest can be individually moved.

A head image acquisition device according to an aspect of the present disclosure may be [4] “the head image acquisition device according to [3], further including a controller that controls operations of the first actuator and the second actuator, wherein the one of the information detection unit and the base is the base, and the controller performs movement control to control the first actuator so that the information detection unit approaches the base in a state in which the second actuator is stopped so that a distance between the information detection unit and the headrest decreases while a position of the headrest relative to the base is maintained at the same position”. In this case, the information detection unit can be brought closer to the headrest while the headrest is maintained at the same position.

A head image acquisition device according to an aspect of the present disclosure may be [5] “the head image acquisition device according to [3], further including a controller that controls operations of the first actuator and the second actuator, wherein the one of the information detection unit and the base is the information detection unit, and the controller performs movement control to control the second actuator so that the headrest approaches the information detection unit while the controller controls the first actuator so that the information detection unit approaches the base so that a distance between the information detection unit and the headrest decreases while a position of the headrest relative to the base is maintained at the same position”. In this case, the information detection unit can be brought closer to the headrest while the headrest is maintained at the same position.

A head image acquisition device according to an aspect of the present disclosure may be [6] “the head image acquisition device according to any one of [3] to [5], wherein the first actuator and the second actuator are arranged in the base”. In this case, a device configuration can be facilitated compared to, for example, the case where the second actuator is arranged in the information detection unit.

A head image acquisition device according to an aspect of the present disclosure may be [7] “the head image acquisition device according to [3] or [5], wherein the one of the information detection unit and the base is the information detection unit, and the first actuator is arranged in the base, and the second actuator is arranged in the information detection unit”. In this case, for example, when compared to the case where the second actuator is arranged in the base, it is possible to arrange the second actuator close to the headrest, and to simplify a structure for transmitting a driving force of the second actuator to the headrest.

A head image acquisition device according to an aspect of the present disclosure may be [8] “the head image acquisition device according to [1] or [2], further including an actuator for moving the information detection unit relative to the base and moving the headrest relative to the one of the information detection unit and the base”. In this case, the information detection unit and the headrest can be moved by a single actuator.

A head image acquisition device according to an aspect of the present disclosure may be [9] “the head image acquisition device according to [8], further including a first moving mechanism that moves the information detection unit relative to the base by a driving force of the actuator, a second moving mechanism that moves the headrest relative to the one of the information detection unit and the base by a driving force of the actuator, and a switching unit that switches a connection state between the actuator and the second moving mechanism”. In this case, by switching the connection state between the actuator and the second moving mechanism using the switching unit, the information detection unit and the headrest can be individually moved.

A head image acquisition device according to an aspect of the present disclosure may be “the head image acquisition device according to [9], wherein the one of the information detection unit and the base is the base, in a state in which the actuator and the second moving mechanism are connected to each other, the information detection unit is movable relative to the base by the first moving mechanism, and the headrest is movable relative to the base by the second moving mechanism, and in a state in which the actuator and the second moving mechanism are disconnected from each other, the information detection unit is able to approach the base by the first moving mechanism so that a distance between the information detection unit and the headrest decreases while a position of the headrest relative to the base is maintained at the same position”. In this case, the information detection unit can be brought closer to the headrest while the headrest is maintained at the same position.

A head image acquisition device according to an aspect of the present disclosure may be “the head image acquisition device according to [9], wherein the one of the information detection unit and the base is the information detection unit, in a state in which the actuator and the second moving mechanism are disconnected from each other, the information detection unit is movable relative to the base by the first moving mechanism, and in a state in which the actuator and the second moving mechanism are connected to each other, the headrest is able to approach the information detection unit by the second moving mechanism while the information detection unit approaches the base by the first moving mechanism so that a distance between the information detection unit and the headrest decreases while a position of the headrest relative to the base is maintained at the same position”. In this case, the information detection unit can be brought closer to the headrest while the headrest is maintained at the same position.

A head image acquisition device according to an aspect of the present disclosure may be “the head image acquisition device according to any one of [1] to [11], further including a chair connected to the base for the subject to sit on”. In this case, it is possible to acquire an image of the head of the subject sitting on the chair.

A head image acquisition method according to an aspect of the present disclosure is “a head image acquisition method using the head image acquisition device according to any one of [1] to [12], the one of the information detection unit and the base being the base, the head image acquisition method including, in this order, steps of adjusting a position of the headrest by moving the headrest relative to the base, fixing a head of the subject to the headrest, and positioning the information detection unit and the headrest at positions where an image of the head is able to be acquired by the information detection unit by moving the information detection unit relative to the base”. According to this head image acquisition method, for the reasons mentioned above, it is possible to suppress contact between the headrest and the information detection unit, and to facilitate alignment between the headrest and the information detection unit.

A head image acquisition method according to an aspect of the present disclosure is “a head image acquisition method using the head image acquisition device according to any one of [1] to [12], the one of the information detection unit and the base being the information detection unit, the head image acquisition method including, in this order, steps of adjusting a position of the headrest by moving the information detection unit relative to the base, fixing a head of the subject to the headrest, and positioning the information detection unit and the headrest at positions where an image of the head is able to be acquired by the information detection unit by moving the headrest relative to the information detection unit while moving the information detection unit relative to the base”.

According to this head image acquisition method, for the reasons mentioned above, it is possible to suppress contact between the headrest and the information detection unit, and to facilitate alignment between the headrest and the information detection unit.

### Advantageous Effects of Invention

According to one aspect of the present disclosure, it is possible to provide a head image acquisition device and a head image acquisition method capable of suppressing contact between a headrest and an information detection unit and facilitating alignment between the headrest and the information detection unit.

### BRIEF DESCRIPTION OF DRAWINGS

FIG. 1 is a side view of a head image acquisition device according to an embodiment.

FIGS. 2(a) and 2(b) are diagrams for describing a head image acquisition method using the head image acquisition device.

FIGS. 3(a) and 3(b) are diagrams for describing the head image acquisition method using the head image acquisition device.

FIG. 4 is a side view of a head image acquisition device of a first modified example.

FIGS. 5(a) and 5(b) are diagrams for describing a head image acquisition method using the head image acquisition device of the first modified example.

FIGS. 6(a) and 6(b) are diagrams for describing the head image acquisition method using the head image acquisition device of the first modified example.

FIG. 7 is a side view of a head image acquisition device of a second modified example.

FIGS. 8(a) and 8(b) are diagrams for describing a head image acquisition method using the head image acquisition device of the second modified example.

FIGS. 9(a) and 9(b) are diagrams for describing the head image acquisition method using the head image acquisition device of the second modified example.

FIG. 10 is a side view of a head image acquisition device of a third modified example.

FIGS. 11(a) and 11(b) are diagrams for describing a head image acquisition method using the head image acquisition device of the third modified example.

FIGS. 12(a) and 12(b) are diagrams for describing the head image acquisition method using the head image acquisition device of the third modified example.

FIG. 13 is a side view of a head image acquisition device of a fourth modified example.

FIGS. 14(a) and 14(b) are diagrams for describing a head image acquisition method using the head image acquisition device of the fourth modified example.

FIGS. 15(a) and 15(b) are diagrams for describing the head image acquisition method using the head image acquisition device of the fourth modified example.

### DESCRIPTION OF EMBODIMENTS

Embodiments of the present disclosure will be described in detail below with reference to the drawings. In each figure, the same or corresponding parts are denoted by the same reference symbols, and redundant description will be omitted.

### [Head Image Acquisition Device]

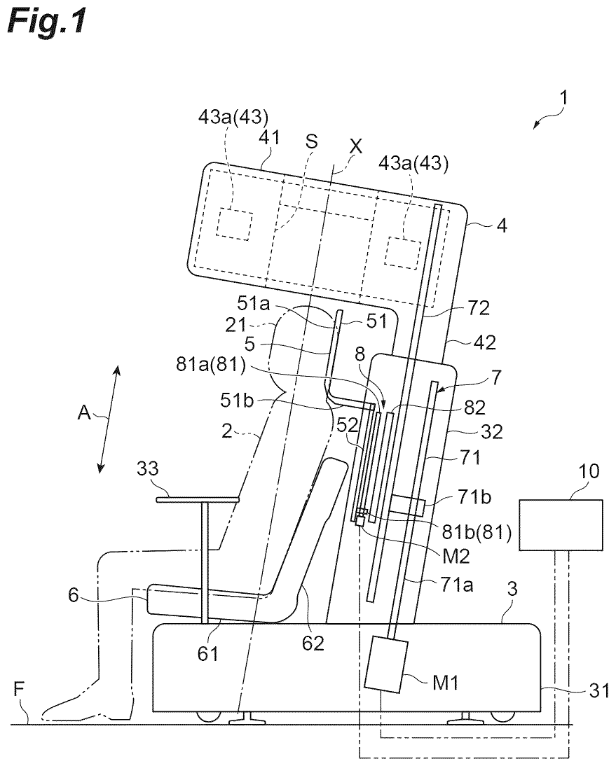

A head image acquisition device 1 illustrated in FIG. 1 is a device for acquiring an image of a head 21 of a subject 2 in a sitting state. In this example, the head image acquisition device 1 is configured as a PET (positron emission tomography) device, and acquires an image by detecting radiation emitted from the head 21 by administering a radioactive drug. The acquired image can be used, for example, for a predetermined diagnosis (checkup). That is, the head image acquisition device 1 can also be regarded as a head image diagnostic device. The head image acquisition device 1 includes a base 3, an information detection unit 4, a headrest 5, and a chair 6. The whole including the base 3, the information detection unit 4, the headrest 5 and the chair 6 may also be referred to as a gantry. The base 3 is a base member including a bottom 31 and a wall 32. Each of the bottom 31 and the wall 32 is formed, for example, in the shape of a rectangular parallelepiped. The bottom 31 is arranged on a floor surface F, which is an installation surface of the head image acquisition device 1. The wall 32 extends vertically upward along a direction A from the bottom 31. In this example, the direction A is inclined with respect to a vertical direction. An inclination angle of the direction A with respect to the vertical direction is, for example, about 10 degrees. An armrest 33 is fixed to the bottom 31. The armrest 33 can be used by the subject 2 sitting on the chair 6 to rest an elbow thereon.

The information detection unit 4 includes a main body 41 and a leg 42. The main body 41 is formed in a cylindrical shape (ring shape), and defines a columnar space S by an inner peripheral surface. An extending direction of an axis X of the main body 41 (an axis of the space S) is parallel to the direction A. The head 21 of the subject 2 is arranged in the space S. A detection unit 43 is arranged inside the main body 41.

The detection unit 43 includes, for example, a plurality of detectors 43a arranged at equal intervals along a circumferential direction so as to surround the space S. In this example, each detector 43a detects radiation emitted from the head 21 of the subject 2 (detects information for acquiring an image of the head 21). Each detector 43a includes, for example, a scintillator that converts radiation into scintillation light and a light receiving element (for example, a photomultiplier tube) that detects the scintillation light. In this way, the main body 41 is included in a detector ring that detects radiation.

The leg 42 is formed, for example, in a rectangular parallelepiped shape, and extends vertically downward along the direction A from the main body 41. A vertically lower portion of the leg 42 is arranged inside the wall 32 of the base 3. The leg 42 is connected to the wall 32 via a first moving mechanism 7, which will be described later, and is movable along the direction A relative to the wall 32.

The headrest 5 has a fixing portion 51 to which the head 21 of the subject 2 is fixed, and a support 52 that supports the fixing portion 51. The fixing portion 51 is formed into a substantially L-shape in a side view (FIG. 1). More specifically, the fixing portion 51 includes a first plate 51a extending along the direction A, and a second plate 51b extending perpendicularly to the first plate 51a from a vertically lower edge of the first plate 51a. The head 21 of the subject 2 is fixed to the first plate 51a. This fixing method may be any method, but for example, the head 21 may be fixed to the first plate 51a using a fixing device such as a net bandage or a headgear. An allowable checkup range may be shown on the first plate 51a to allow identification by the subject 2 or a checkup worker (technician). In this case, for example, by fixing the head 21 to the first plate 51a so that the head 21 is located within the allowable checkup range, checkup can be reliably carried out, and alignment between the head 21 relative to the first plate 51a can be facilitated.

The support 52 is formed into a plate shape, for example, and extends vertically downward along the direction A from an edge of the second plate 51b on the opposite side from the first plate 51a. A vertically lower portion of the support 52 is arranged inside the wall 32 of the base 3. The support 52 is connected to the wall 32 via a second moving mechanism 8, which will be described later, and is movable along the direction A relative to the wall 32.

The chair 6 is used for the subject 2 to sit on. The chair 6 has a seat portion 61 and a back portion 62. The back portion 62 extends vertically upward from the seat portion 61. As illustrated in FIG. 1, during information detection for image acquisition, the subject 2 sits on the seat portion 61 so that a back of the subject 2 touches the back portion 62. In this example, the chair 6 is connected to the bottom 31 of the base 3 so that the chair 6 can horizontally move along a front-rear direction (a direction that intersects the direction A). Note that, when a side where the chair 6 is positioned relative to the wall 32 of the base 3 is defined as a front side, the back portion 62 is arranged on a rear side relative to the seat portion 61.

The head image acquisition device 1 further includes a first motor (first actuator) M1, the first moving mechanism 7, a second motor (second actuator) M2, the second moving mechanism 8, and a controller 10. The first motor M1 is used to move the information detection unit 4 relative to the base 3, and the second motor M2 is used to move the headrest 5 relative to the base 3. The first moving mechanism 7 is a mechanism for moving the information detection unit 4 relative to the base 3 by a driving force of the first motor M1, and the second moving mechanism 8 is a mechanism for moving the headrest 5 relative to the base 3 by a driving force of the second motor M2. Note that, even though the first motor M1, the first moving mechanism 7, the second motor M2, and the second moving mechanism 8 are arranged inside the base 3 or the information detection unit 4 as described below, these elements are indicated by solid lines in each figure for convenience of description.

The first motor M1 is arranged inside the bottom 31 of the base 3. The first moving mechanism 7 includes a first ball screw 71 and a first guide rail 72. The first ball screw 71 is arranged inside the wall 32 of the base 3 and is fixed to the wall 32. The first ball screw 71 includes a screw shaft 71a and a nut 71b. The screw shaft 71a extends along the direction

A. The screw shaft 71a is connected to the first motor M1, and is rotated by a driving force of the first motor M1. The nut 71b is screwed onto the screw shaft 71a, and moves along the screw shaft 71a (along the direction A) in accordance with rotation of the screw shaft 71a. The first guide rail 72 extends along the direction A. The first guide rail 72 is connected to the nut 71b and moves along the direction A together with the nut 71b. The first guide rail 72 is arranged inside the leg 42 of the information detection unit 4 and is fixed to the leg 42. Therefore, when the nut 72b moves along the direction A due to a driving force of the first motor M1, the information detection unit 4 moves along the direction A relative to the base 3. In this way, the information detection unit 4 is movable along the direction A relative to the base 3 by the driving force of the first motor M1. Note that a rotation direction of the first motor M1 is switchable, and the information detection unit 4 can move toward or away from the base 3 depending on the rotation direction of the first motor M1.

The second motor M2 is arranged inside the wall 32 of the base 3. The second moving mechanism 8 includes a second ball screw 81 and a second guide rail 82. The second ball screw 81 is arranged inside the wall 32 and is fixed to the wall 32. The second ball screw 81 includes a screw shaft 81a and a nut 81b. The screw shaft 81a extends along the direction A. The screw shaft 81a is connected to the second motor M2, and is rotated by a driving force of the second motor M2. The nut 81b is screwed onto the screw shaft 81a, and moves along the screw shaft 81a (along the direction A) in accordance with rotation of the screw shaft 81a. The second guide rail 82 extends along the direction A and guides a moving direction of the nut 81b. The support 52 of the headrest 5 is connected to nut 81b. In this way, the headrest 5 is movable along the direction A together with the nut 81b, and the headrest 5 is movable along the direction A relative to the base 3 by a driving force of the second motor M2. Note that a rotation direction of the second motor M2 is switchable, and the headrest 5 can move toward or away from the base 3 depending on the rotation direction of the second motor M2.

In this way, in the head image acquisition device 1, a direction in which the information detection unit 4 moves relative to the base 3 and a direction in which the headrest 5 moves relative to the base 3 are along the direction A (that is, parallel to the direction A), and are parallel to each other. Further, the information detection unit 4 can only move along the direction A relative to the base 3, and does not move so that an angle between the information detection unit 4 and the base 3 changes. Similarly, the headrest 5 can only move along the direction A relative to the base 3, and does not move so that an angle between the headrest 5 and the base 3 changes.

For example, the controller 10 includes a computer including a processor (CPU), and a RAM and a ROM (storage unit) which are recording media. The controller 10 controls an operation of each unit of the head image acquisition device 1. For example, the controller 10 controls operations of the first motor M1 and the second motor M2. For example, the head image acquisition device 1 includes a reception unit for receiving an instruction (operation) from the checkup worker, and the controller 10 controls operations of the first motor M1 and the second motor M2 based on an instruction received by the reception unit. The reception unit may include, for example, an operation button, etc. provided on the base 3.

### [Head Image Acquisition Method]

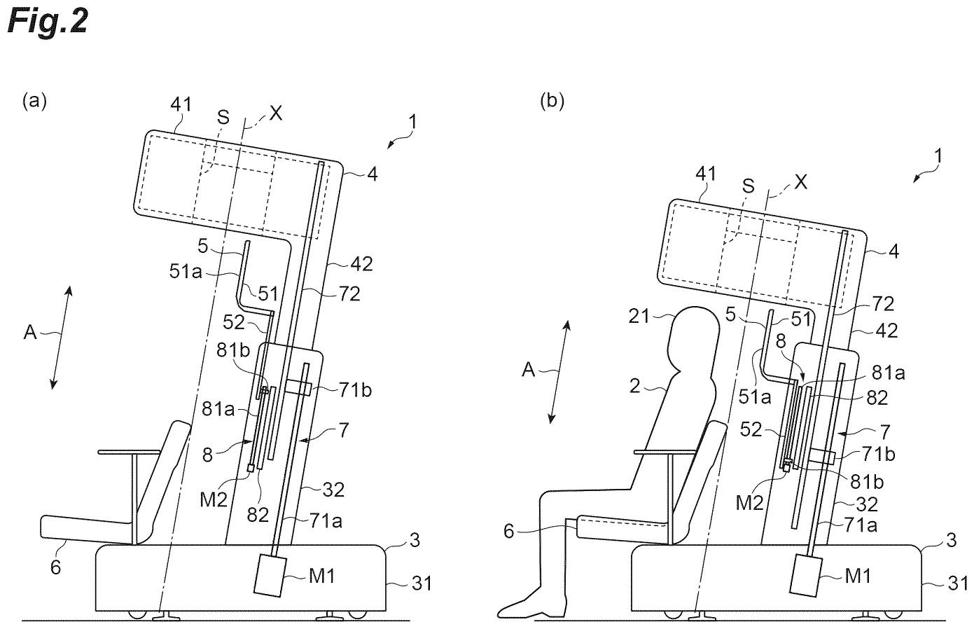

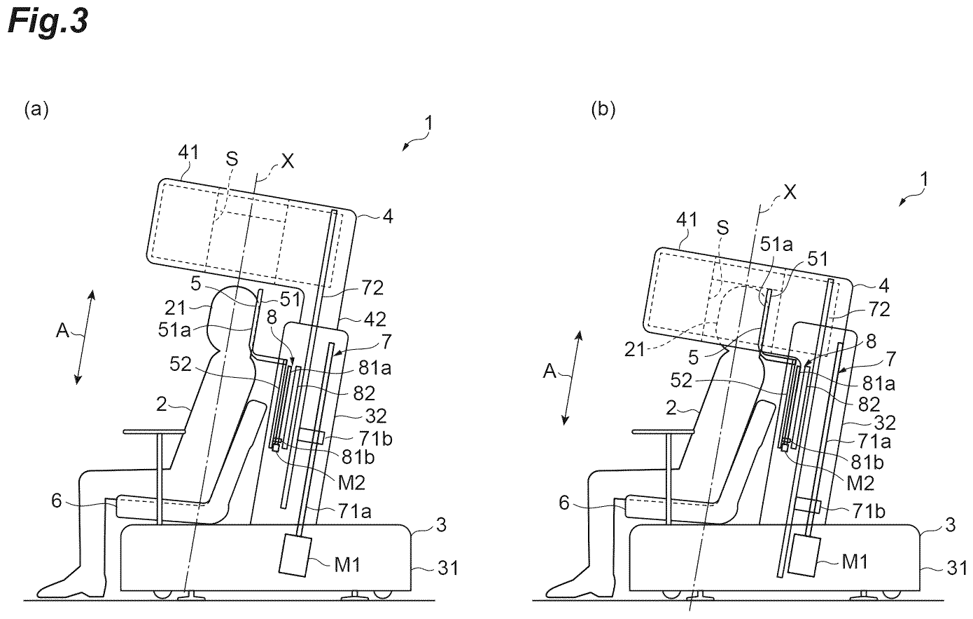

An example of a head image acquisition method using the head image acquisition device 1 will be described with reference to FIGS. 2 and 3. An operation of the head image acquisition device 1 in a description below is performed by, for example, the controller 10 controlling each unit based on an instruction from the checkup worker.

FIG. 2(a) illustrates the head image acquisition device 1 in an initial state before starting checkup. From the initial state, first, as illustrated in FIG. 2(b), the subject 2 sits on the chair 6. Subsequently, a first adjustment step of adjusting a position of the headrest 5 is performed. In the first adjustment step, the position of the headrest 5 is adjusted by moving the headrest 5 along the direction A relative to the base 3 using a driving force of the second motor M2. For example, the headrest 5 is moved vertically downward along the direction A from the initial position illustrated in FIG. 2(a), and the headrest 5 is stopped at a position where a height of the first plate 51a of the headrest 5 matches the head 21 of the subject 2 as illustrated in FIG. 2(b). In this way, in the first adjustment step, the position of the headrest 5 is adjusted so that the position corresponds to a position of the head 21 of the subject 2 sitting on the chair 6. Note that, even though the information detection unit 4 moves relative to the base 3 (moves together with the headrest 5) in the first adjustment step in an example illustrated in FIG. 2(b), the information detection unit 4 does not have to move relative to the base 3 in the first adjustment step.

Subsequently, as illustrated in FIG. 3(a), the chair 6 moves to the rear side (to the wall 32 side of the base 3). Subsequently, a fixing step is performed to fix the head 21 of the subject 2 to the headrest 5. In the fixing step, as described above, for example, the head 21 is fixed to the first plate 51a using a fixing device such as a net bandage or headgear.

Subsequently, as illustrated in FIG. 3(b), a second adjustment step is performed to adjust the position of the information detection unit 4. In the second adjustment step, the information detection unit 4 is moved along the direction A relative to the base 3 by a driving force of the first motor M1, so that the information detection unit 4 and the headrest 5 are positioned at positions where an image of the head 21 can be acquired by the information detection unit 4. In this example, when the information detection unit 4 moves vertically downward along the direction A, the information detection unit 4 approaches the headrest 5 (a distance between the information detection unit 4 and the headrest 5 decreases), and the information detection unit 4 and the headrest 5 are positioned at checkup positions. The checkup positions are positions where the head 21 fixed to the headrest 5 is arranged in a space S of the information detection unit 4.

In the second adjustment step, the second motor M2 is stopped and the position of the headrest 5 is fixed. That is, in the second adjustment step, the controller 10 performs movement control to control the first motor M1 so that the information detection unit 4 approaches the base 3 in a state in which the second motor M2 is stopped so that a distance between the information detection unit 4 and the headrest 5 decreases while a position of the headrest 5 relative to the base 3 is maintained at the same position. In the second adjustment step, the controller 10 may stop movement of the information detection unit 4 based on an instruction of the checkup worker. However, when the head 21 is arranged in the space S, the controller 10 may automatically stop movement of the information detection unit 4. In the latter case, for example, a sensor for detecting that the head 21 is arranged in the space S may be provided in the information detection unit 4, and the controller 10 may stop movement of the information detection unit 4 based on a detection result of the sensor and position information of the information detection unit 4 and the headrest 5.

After the second adjustment step, an image of the head 21 of the subject 2 is acquired. After image acquisition, the head image acquisition device 1 is returned to the initial state by a process reverse to the above-described process. Specifically, first, the information detection unit 4 moves vertically upward along the direction A and returns to a position illustrated in FIG. 3(a). Subsequently, fixation of the head 21 to the headrest 5 is released. Subsequently, the chair 6 moves forward and returns to the initial position illustrated in FIG. 2(a), and the headrest 5 moves vertically upward along the direction A and returns to the initial position illustrated in FIG. 2(a). Thereafter, the subject 2 gets off the chair 6. Through the above process, the head image acquisition device 1 returns to the initial state.

### [Function and Effect]

In the head image acquisition device 1, the headrest 5 is connected to the base 3 so that the headrest 5 is movable relative to the base 3. In this way, after fixing the head 21 of the subject 2 to the headrest 5, by moving the information detection unit 4 relative to the base 3, the headrest 5 and the information detection unit 4 can be aligned. In the head image acquisition device 1, the headrest 5 is connected to the base 3. Therefore, for example, when compared to the case where the headrest 5 is provided on the chair 6, it is possible to suppress occurrence of a situation in which the headrest 5 and the information detection unit 4 are in contact with each other. In addition, since a relative position between the headrest 5 and the information detection unit 4 can be easily structurally determined, it is possible to facilitate alignment between the headrest 5 (the head 21 of the subject 2) and the information detection unit 4. Therefore, according to the head image acquisition device 1, it is possible to suppress contact between the headrest 5 and the information detection unit 4, and to facilitate alignment between the headrest 5 and the information detection unit 4. In addition, by facilitating alignment between the headrest 5 and the information detection unit 4, it is possible to omit an element for alignment between the headrest 5 and the information detection unit 4 (for example, a floodlight, a camera, etc.).

The direction in which the information detection unit 4 moves relative to the base 3 is parallel to the direction in which the headrest 5 moves relative to the base 3. In this way, contact between the headrest 5 and the information detection unit 4 can be further suppressed.

The head image acquisition device 1 includes the first motor M1 for moving the information detection unit 4 relative to the base 3, and the second motor M2 for moving the headrest 5 relative to the base 3. In this way, the information detection unit 4 and the headrest 5 can be individually moved.

The controller 10 performs movement control to control the first motor M1 so that the information detection unit 4 approaches the base 3 in a state in which the second motor M2 is stopped so that a distance between the information detection unit 4 and the headrest 5 decreases while a position of the headrest 5 relative to the base 3 is maintained at the same position. In this way, the information detection unit 4 can be brought closer to the headrest 5 while the headrest 5 is maintained at the same position.

The first motor M1 and the second motor M2 are arranged in the base 3. In this way, a device configuration can be facilitated compared to, for example, the case where the second motor M2 is arranged in the information detection unit 4.

The head image acquisition device 1 is connected to the base 3 and includes the chair 6 on which the subject 2 sits. As a result, it is possible to acquire an image of the head 21 of the subject 2 in a state of sitting on the chair 6. Furthermore, when a chair is interposed between the headrest and the support body as in Patent Literature 1 mentioned above, contact with the gantry (the information detection unit 4) cannot be suppressed unless movement or position information of the chair is acquired. On the other hand, in the head image acquisition device 1 according to the embodiment, the headrest 5 is connected to the base 3 and is independent from the chair 6, and thus it is possible to facilitate suppression of contact between the headrest 5 and the information detection unit 4.

The head image acquisition method of the embodiment includes a step of adjusting the position of the headrest 5 by moving the headrest 5 relative to the base 3, a step of fixing the head 21 of the subject 2 to the headrest 5, and a step of positioning the information detection unit 4 and the headrest 5 at positions where the information detection unit 4 can acquire an image of the head 21 by moving the information detection unit 4 relative to the base 3 in this order. According to this head image acquisition method, for the reasons mentioned above, it is possible to suppress contact between the headrest 5 and the information detection unit 4, and to facilitate alignment between the headrest 5 and the information detection unit 4.

### MODIFIED EXAMPLES

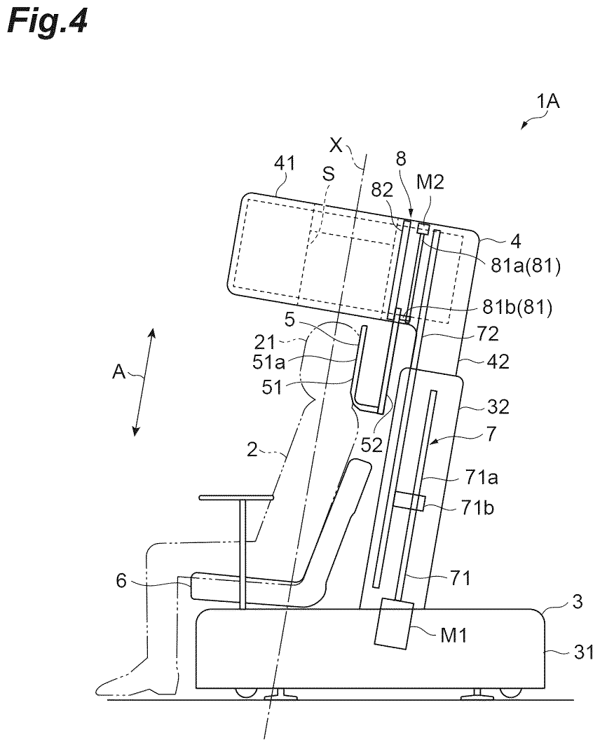

FIG. 4 is a side view of a head image acquisition device 1A of a first modified example. The head image acquisition device 1A has the same configuration as that of the head image acquisition device 1 of the above embodiment except for the following points. In the head image acquisition device 1A, the second motor M2 is arranged inside the main body 41 of the information detection unit 4. The second ball screw 81 is arranged inside the main body 41 and is fixed to the main body 41. In the first modified example, the headrest 5 is fixed to the main body 41 via the second moving mechanism 8, and the headrest 5 can move along the direction A relative to the information detection unit 4 by a driving force of the second motor M2.

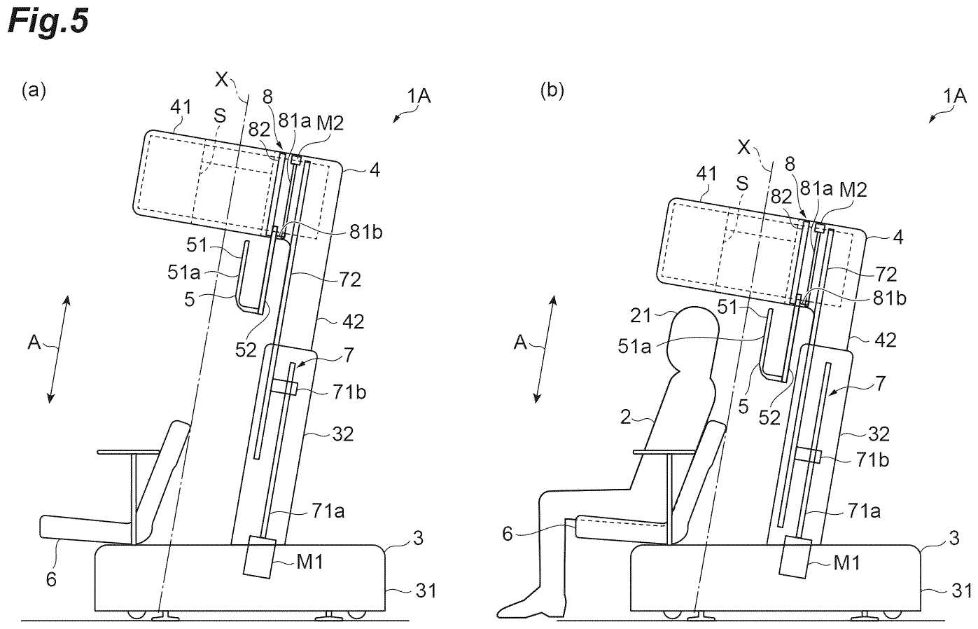

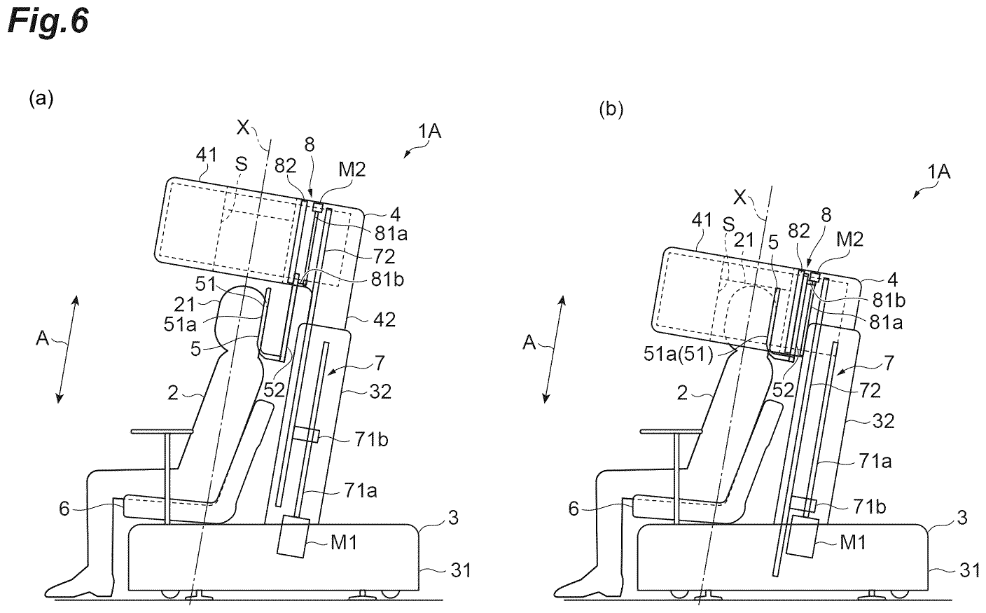

FIGS. 5 and 6 are diagrams for describing an example of a head image acquisition method using the head image acquisition device 1A of the first modified example. In a first adjustment step of the head image acquisition method of the first modified example, the position of the headrest 5 is adjusted by moving the information detection unit 4 along the direction A relative to the base 3 using a driving force of the first motor M1 (FIG. 5(a) and FIG. 5(b)). In this instance, the headrest 5 moves together with the information detection unit 4. For example, the information detection unit 4 is moved vertically downward along the direction A from an initial position illustrated in FIG. 5(a), and the information detection unit 4 is stopped at a position where the height of the first plate 51a of the headrest 5 matches the head 21 of the subject 2 as illustrated in FIG. 5(b). In this way, the position of the headrest 5 is adjusted so that the position corresponds to the position of the head 21 of the subject 2 sitting on the chair 6.

In a second adjustment step of the head image acquisition method of the first modified example, the information detection unit 4 and the headrest 5 are positioned at positions where the information detection unit 4 can acquire an image of the head 23 by moving the headrest 5 relative to the information detection unit 4 by a driving force of the second motor M2 while moving the information detection unit 4 relative to the base 3 by a driving force of the first motor M1 (FIG. 6(a) and FIG. 6(b)). In this example, in parallel with the information detection unit 4 moving along the direction A and approaching the base 3, the headrest 5 moves along the direction A and approaching the information detection unit 4 (the distance between headrest 5 and the information detection unit 4 decreases). A moving speed of the information detection unit 4 is the same as a moving speed of the headrest 5. That is, the controller 10 controls the first motor M1 and the second motor M2 so that the moving speed of the information detection unit 4 is the same as the moving speed of the headrest 5. As a result, the information detection unit 4 moves vertically downward along the direction A while the position of the headrest 5 relative to the base 3 is maintained at the same position. That is, in the second adjustment step of the first modified example, the controller 10 performs movement control to control the second motor M2 so that the headrest 5 approaches the information detection unit 4 while the controller 10 controls the first motor M1 so that the information detection unit 4 approaches the base 3 so that the distance between the information detection unit 4 and the headrest 5 decreases while the position of the headrest 5 relative to the base 3 is maintained at the same position.

With this first modified example, as in the above embodiment, it

is possible to suppress contact between the headrest 5 and the information detection unit 4, and to facilitate alignment between the headrest 5 and the information detection unit 4.

Further, in the first modified example, the controller 10 performs movement control to control the second motor M2 so that the headrest 5 approaches the information detection unit 4 while the controller 10 controls the first motor M1 so that the information detection unit 4 approaches the base 3 so that the distance between the information detection unit 4 and the headrest 5 decreases while the position of the headrest 5 relative to the base 3 is maintained at the same position. In this way, the information detection unit 4 can be brought closer to the headrest 5 while the headrest 5 is maintained at the same position.

Further, in the first modified example, the first motor M1 is arranged in the base 3, and the second motor M2 is arranged in the information detection unit 4. In this way, for example, when compared to the case where the second motor M2 is arranged in the base 3, it is possible to arrange the second motor M2 close to the headrest 5, and to simplify a structure for transmitting a driving force of the second motor M2 to the headrest 5.

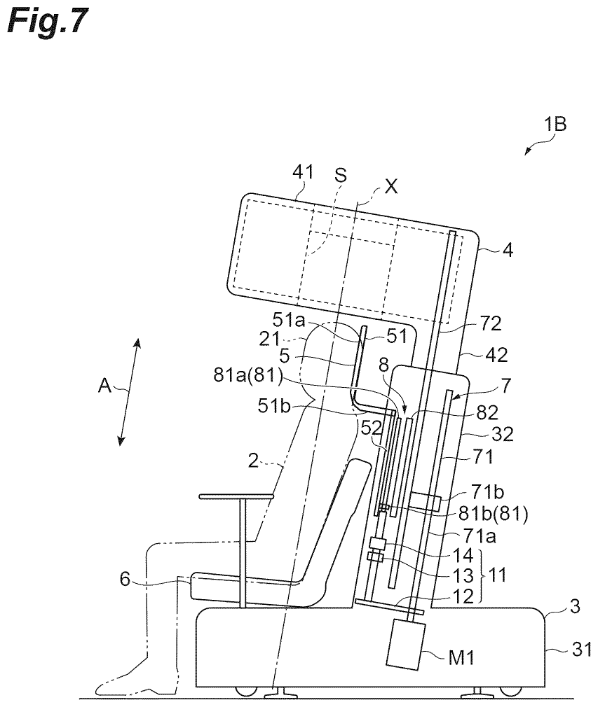

FIG. 7 is a side view of a head image acquisition device 1B of a second modified example. The head image acquisition device 1B has the same configuration as that of the head image acquisition device 1 of the above embodiment except for the following points. The head image acquisition device 1B includes only the first motor M1 and does not include the second motor M2. In the second modified example, the first motor M1 is used to move the information detection unit 4 relative to the base 3 and to move the headrest 5 relative to the base 3. The first moving mechanism 7 moves the information detection unit 4 relative to the base 3 by a driving force of the first motor M1, and the second moving mechanism 8 moves the headrest 5 relative to the base 3 by a driving force of the first motor M1.

The head image acquisition device 1B includes a switching unit 11 that switches a connection state between the first motor M1 and the second moving mechanism 8. In this example, the switching unit 11 includes a power transmission mechanism 12, a clutch 13, and a brake 14 that prevents the headrest 5 from falling, and is arranged in the base 3. The power transmission mechanism 12 is connected to the first motor M1, branches a driving force of the first motor M1, and transmits a part of the driving force to the second moving mechanism 8. The power transmission mechanism 12 includes, for example, a plurality of gears. The clutch 13 is connected between the first motor M1 and the second moving mechanism 8, and switches the connection state between the first motor M1 and the second moving mechanism 8 between a first state in which the first motor M1 and the second moving mechanism 8 are connected to each other and a second state in which the first motor M1 and the second moving mechanism 8 are disconnected from each other. In the first state, the driving force of the first motor M1 is transmitted to the second moving mechanism 8, while in the second state, the driving force of the first motor M1 is not transmitted to the second moving mechanism 8. The clutch 13 is, for example, a mechanical clutch, but may be any type of clutch. An operation of the clutch 13 (switching unit 11) is controlled, for example, according to an operation of the checkup worker, but may also be controlled by the controller 10. In this example, the clutch 13 is connected to the second moving mechanism 8 via the brake 14. Note that the brake 14 may be omitted.

In the first state, since the driving force of the first motor MI is transmitted to both the first moving mechanism 7 and the second moving mechanism 8, the information detection unit 4 is movable relative to the base 3 by the first moving mechanism 7, and the headrest 5 is movable relative to the base 3 by the second moving mechanism 8. The moving direction of the information detection unit 4 is the same as a moving direction of the headrest 5. That is, when the information detection unit 4 moves vertically downward to approach the base 3, the headrest 5 also moves vertically downward to approach the base 3. When the information detection unit 4 moves vertically upward away from the base 3, the headrest 5 also moves vertically upward away from the base 3. Further, a moving speed of the information detection unit 4 is the same as a moving speed of the headrest 5. In this way, the information detection unit 4 and the headrest 5 can integrally move while maintaining a positional relationship therebetween. In the second state, since the driving force of the first motor M1 is transmitted only to the first moving mechanism 7, the information detection unit 4 is movable relative to the base 3 by the first moving mechanism 7, but the position of the headrest 5 is fixed.

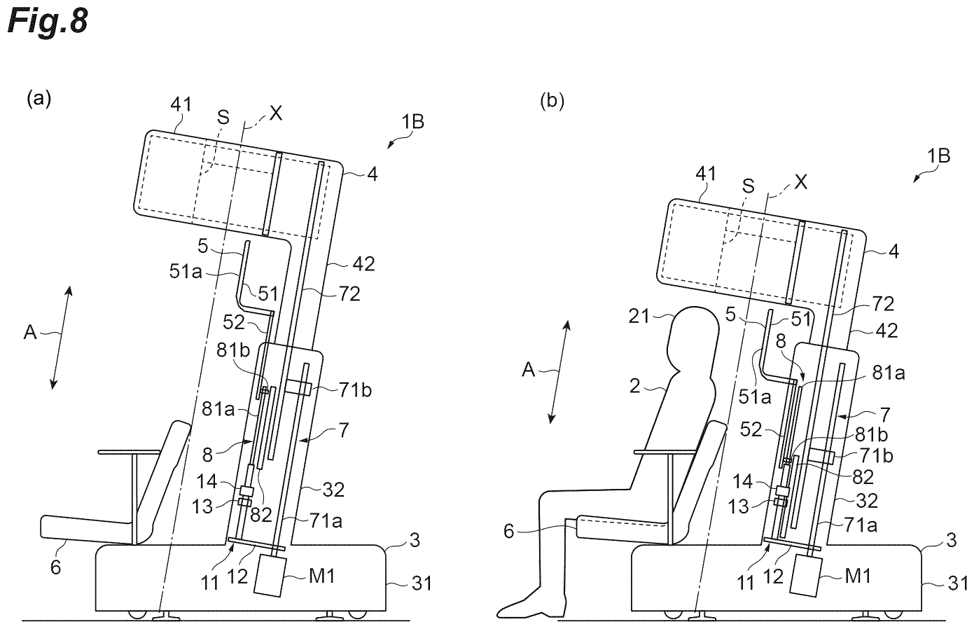

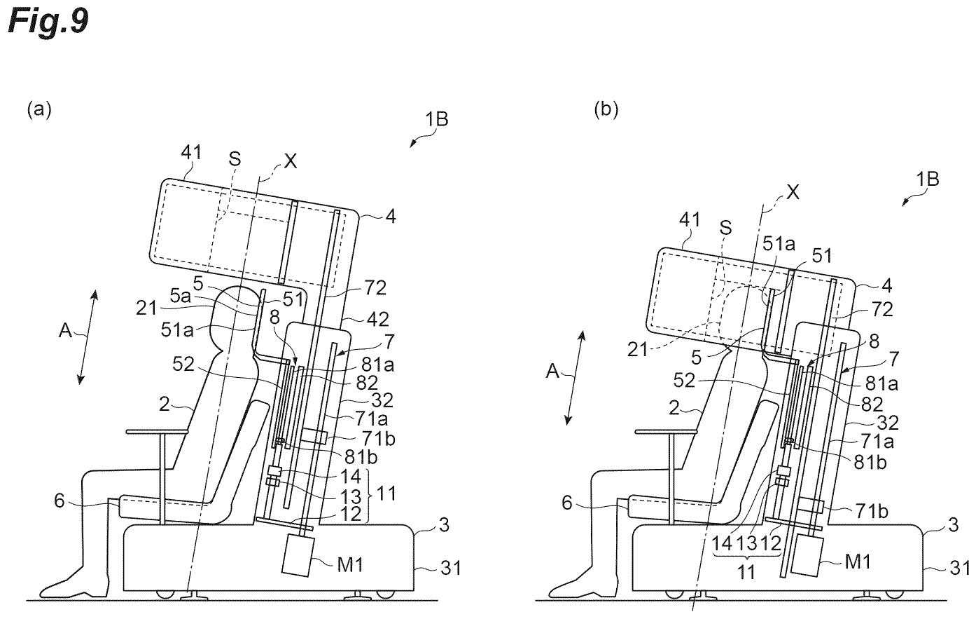

FIGS. 8 and 9 are diagrams for describing an example of a head image acquisition method using the head image acquisition device 1B of the second modified example. In a first adjustment step of the head image acquisition method of the second modified example, the connection state between the first motor M1 and the second moving mechanism 8 is set to a first state, and the first motor M1 is connected to both the first moving mechanism 7 and the second moving mechanism 8. Then, the position of the headrest 5 is adjusted by moving the headrest 5 together with the information detection unit 4 along the direction A relative to the base 3 by the driving force of the first motor M1 (FIGS. 8(a) and 8(b)). For example, the headrest 5 is moved together with the information detection unit 4 vertically downward along the direction A from an initial position illustrated in FIG. 8(a), the headrest 5 is stopped at a position where the height of the first plate 51a of the headrest 5 matches the head 21 of the subject 2 as illustrated in FIG. 8(b).

In a second adjustment step of the head image acquisition method of the second modified example, the connection state between the first motor M1 and the second moving mechanism 8 is set to a second state, and the first motor M1 is connected only to the first moving mechanism 7. Then, by moving the information detection unit 4 along the direction A relative to the base 3 by the driving force of the first motor M1, the information detection unit 4 and the headrest 5 are positioned at positions where the information detection unit 4 can acquire an image of the head 21 (FIGS. 9(a) and 9(b)). In this example, when the information detection unit 4 moves vertically downward along the direction A, the information detection unit 4 approaches the headrest 5 (the distance between the information detection unit 4 and the headrest 5 decreases), and the information detection unit 4 and the headrest 5 are located at checkup positions (space S). In the second adjustment step, the first motor M1 is not connected to the second moving mechanism 8, and the position of the headrest 5 is fixed. That is, in the second adjustment step, the information detection unit 4 can approach the base 3 by the first moving mechanism 7 so that the distance between the information detection unit 4 and the headrest 5 is decreased while the position of the headrest 5 relative to the base 3 is maintained at the same position.

With such a second modified example, as in the above embodiment, it is possible to suppress contact between the headrest 5 and the information detection unit 4, and to facilitate alignment between the headrest 5 and the information detection unit 4.

Further, in the second modified example, the information detection unit 4 is moved relative to the base 3 and the headrest 5 is moved relative to the base 3 by the first motor M1. In this way, in the second modified example, the information detection unit 4 and the headrest 5 can be moved by a single actuator (the first motor M1).

Furthermore, the second modified example includes the switching unit 11 that switches the connection state between the first motor M1 and the second moving mechanism 8. Therefore, by switching the connection state between the first motor M1 and the second moving mechanism 8 using the switching unit 11, the information detection unit 4 and the headrest 5 can be individually moved.

Further, in the second modified example, in the first state in which the first motor M1 and the second moving mechanism 8 are connected to each other, the information detection unit 4 is movable relative to the base 3 by the first moving mechanism 7, and the headrest 5 is movable relative to the base 3 by the second moving mechanism 8, and in the second state in which the first motor M1 and the second moving mechanism 8 are disconnected from each other, the information detection unit 4 can approach the base 3 by the first moving mechanism 7 so that the distance between the information detection unit 4 and the headrest 5 decreases while the position of the headrest 5 relative to the base 3 is maintained at the same position. In this way, the information detection unit 4 can be brought closer to the headrest 5 while the headrest 5 is maintained at the same position.

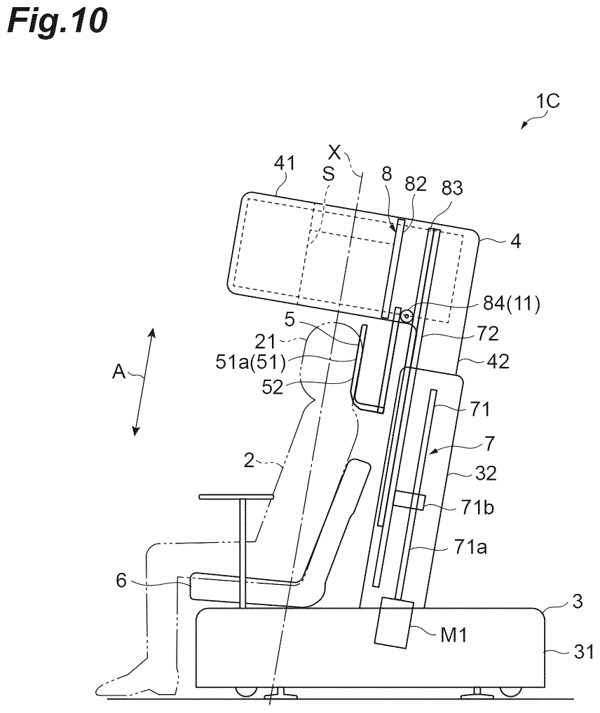

FIG. 10 is a side view of a head image acquisition device 1C of a third modified example. The head image acquisition device 1C has the same configuration as that of the head image acquisition device 1B of the second modified embodiment except for the following points. In the head image acquisition device 1C, the second moving mechanism 8 is arranged in the main body 41 of the information detection unit 4. The headrest 5 is fixed to the main body 41 via the second moving mechanism 8, and the headrest 5 is movable along the direction A relative to the information detection unit 4 by a driving force of the first motor M1.

The second moving mechanism 8 includes a second guide rail 82, a rack 83, and a pinion 84. The rack (first rack) 83 extends along the direction A. The rack 83 is formed so that a row of teeth meshing with teeth of the pinion 84 is aligned in the direction A. The pinion 84 is a cylindrical gear including teeth on an outer peripheral surface. In this example, a row of teeth meshing with the teeth of the pinion 84 is formed to be aligned in the direction A on the support 52 of the headrest 5, and the pinion 84 is arranged between the rack 83 and the support 52 to engage with the rack 83 and the support 52. That is, in this example, the support 52 functions as a second rack, and in this case, the support 52 can also be considered to be a part of the second moving mechanism 8. As the pinion 84 rotates, the pinion 84 moves along the direction A on the rack 83 together with the support 52, and as the support 52 moves together with the pinion 84, the headrest 5 moves along the direction A. In this example, the second guide rail 82 guides a moving direction of the pinion 84.

A pinion shaft of the pinion 84 is provided with a clutch functioning as the switching unit 11 that switches the connection state between the first motor M1 and the second moving mechanism 8. For example, this clutch is configured similarly to the clutch 13 described above, and is connected between the first motor M1 and the pinion 84 (the second moving mechanism 8). This clutch switches a connection state between the first motor M1 and the pinion 84 between a first state in which the first motor M1 and the pinion 84 are connected to each other and a second state in which the first motor M1 and the pinion 84 are disconnected from each other. In the first state, a driving force of the first motor M1 is transmitted to the pinion 84, while in the second state, the driving force of the first motor M1 is not transmitted to the pinion 84. The second moving mechanism 8 has a power transmission mechanism that branches a driving force of the first motor M1 and transmits a part of the driving force to the pinion 84, but illustration of the power transmission mechanism is omitted in FIGS. 10 to 12.

In the second state, the driving force of the first motor M1 is transmitted only to the first moving mechanism 7, so that the information detection unit 4 is movable relative to the base 3 by the first moving mechanism 7. In this instance, the headrest 5 can integrally move together with the information detection unit 4. In the first state, the driving force of the first motor M1 is transmitted to both the first moving mechanism 7 and the second moving mechanism 8, so that the information detection unit 4 is movable relative to the base 3 by the first moving mechanism 7, and the headrest 5 is movable relative to the information detection unit 4 by the second moving mechanism 8. The moving direction of the information detection unit 4 is opposite to the moving direction of the headrest 5. That is, when the information detection unit 4 moves vertically downward to approach the base 3, the headrest 5 moves vertically upward to approach the information detection unit 4. When the information detection unit 4 moves vertically upward away from the base 3, the headrest 5 moves vertically downward away from the information detection unit 4. Further, the moving speed of the information detection unit 4 is the same as the moving speed of the headrest 5. In this way, in the first state, the headrest 5 can approach the information detection unit 4 while the information detection unit 4 approaches the base 3 while the position of the headrest 5 relative to the base 3 is maintained at the same position.

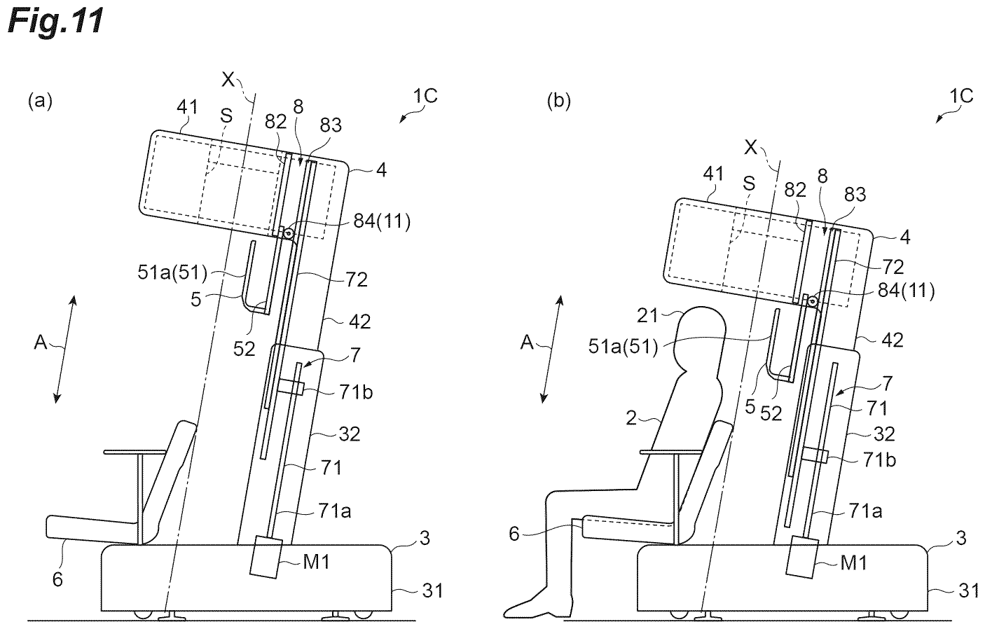

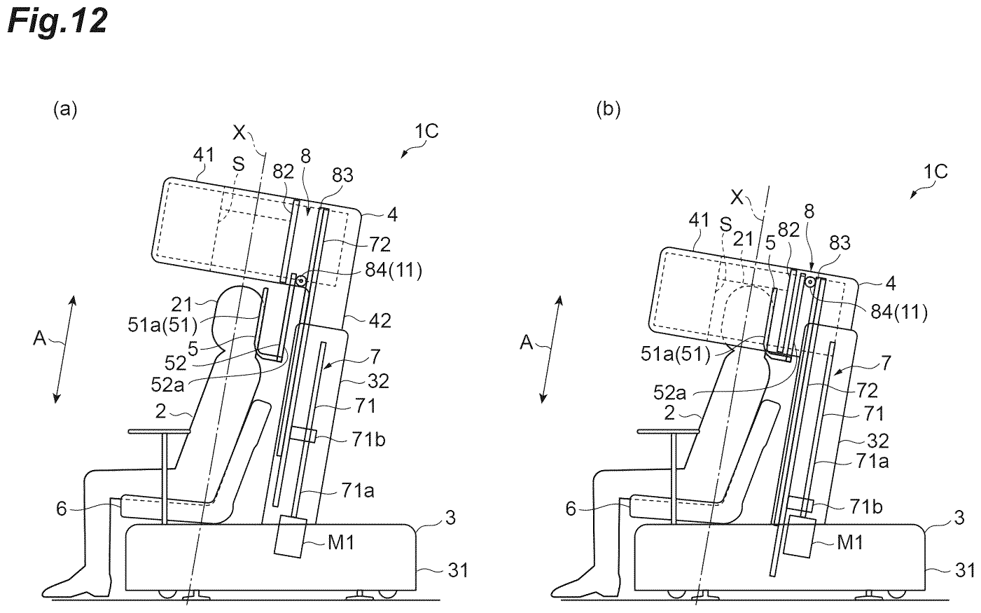

FIGS. 11 and 12 are diagrams for describing an example of a head image acquisition method using the head image acquisition device 1C of the third modified example. In a first adjustment step of the head image acquisition method of the third modified example, the connection state between the first motor M1 and the second moving mechanism 8 is set to a second state, and the first motor M1 is connected only to the first moving mechanism 7. Then, the position of the headrest 5 is adjusted by moving the information detection unit 4 together with the headrest 5 along the direction A relative to the base 3 using a driving force of the first motor M1 (FIGS. 11(a) and 11(b)). For example, the information detection unit 4 and the headrest 5 are moved vertically downward along the direction A from an initial position illustrated in FIG. 11(a), and the information detection unit 4 is stopped at a position where the height of the plate 51a of the headrest 5 matches the head 21 of the subject 2 as illustrated in FIG. 11(b).

In the second adjustment step of the head image acquisition method of the third modified example, the connection state between the first motor M1 and the second moving mechanism 8 is set to the first state, and the first motor M1 is connected to both the first moving mechanism 7 and the second moving mechanism 8. Then, by moving the headrest 5 relative to the information detection unit 4 while moving the information detection unit 4 along the direction A relative to the base 3 by a driving force of the first motor M1, the information detection unit 4 and the headrest 5 are positioned at positions where an image of the head 21 can be acquired by the information detection unit 4 (FIGS. 12(a) and 12(b)). In this example, as described above, when the information detection unit 4 moves vertically downward along the direction A while the position of the headrest 5 relative to the base 3 is maintained at the same position, the information detection unit 4 approaches the headrest 5 (the distance between the information detection unit 4 and the headrest 5 decreases), and the information detection unit 4 and the headrest 5 are positioned at the checkup positions (space S). That is, in the second adjustment step, the headrest 5 can be brought closer to the information detection unit 4 by the second moving mechanism 8 while the information detection unit 4 approaches the base 3 by the first moving mechanism 7 so that the distance between the information detection unit 4 and the headrest 5 decreases while the position of the headrest 5 relative to the base 3 is maintained at the same position.

With this third modified example, similar to the above embodiment, it is possible to suppress contact between the headrest 5 and the information detection unit 4, and to facilitate alignment between the headrest 5 and the information detection unit 4.

Furthermore, in the third modified example, in the second state in which the first motor M1 and the second moving mechanism 8 are not connected to each other, the information detection unit 4 is movable relative to the base 3 by the first moving mechanism 7, and in the first state in which the first motor M1 and the second moving mechanism 8 are connected from each other, the headrest 5 can be brought closer to the information detection unit 4 by the second moving mechanism 8 while the information detection unit 4 approaches the base 3 by the first moving mechanism 7 so that the distance between the information detection unit 4 and the headrest 5 decreases while the position of the headrest 5 relative to the base 3 is maintained at the same position. In this way, the information detection unit 4 can be brought closer to the headrest 5 while the headrest 5 is maintained at the same position.

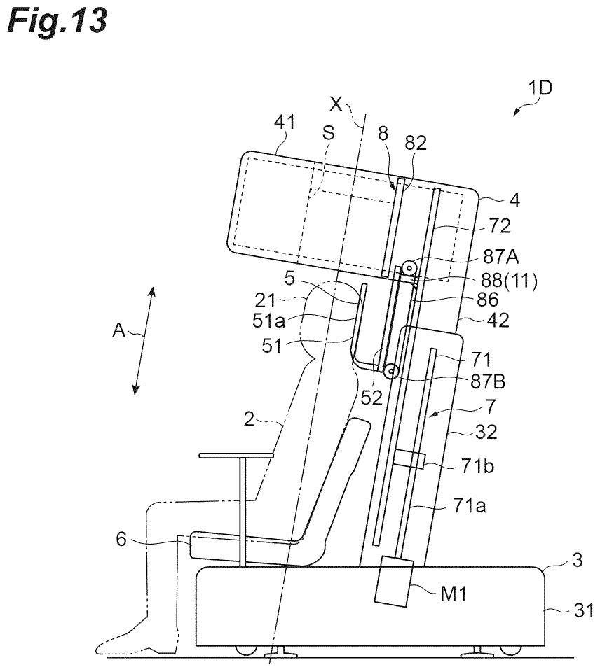

FIG. 13 is a side view of a head image acquisition device ID of a fourth modified example. The head image acquisition device 1D has the same configuration as that of the head image acquisition device 1C of the third modified example except for the following points. In the head image acquisition device 1D, a second moving mechanism 8 includes a second guide rail 82, a belt 86, a pair of pulleys 87A and 87B, and a clutch 88. The belt 86 is an endless belt, and is spanned between the pair of pulleys 87A and 87B and is rotatably supported. The support 52 of the headrest 5 is fixed to the belt 86. The belt 86 rotates in accordance with rotation of the pulleys 87A and 87B. Rotation of the belt 86 moves the support 52, and as a result, the headrest 5 moves along the direction A.

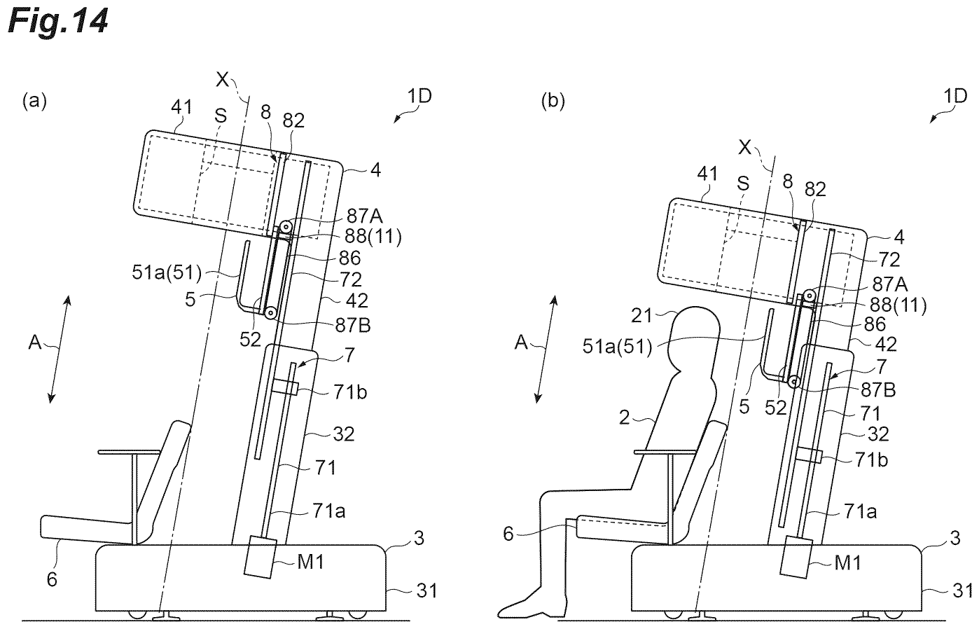

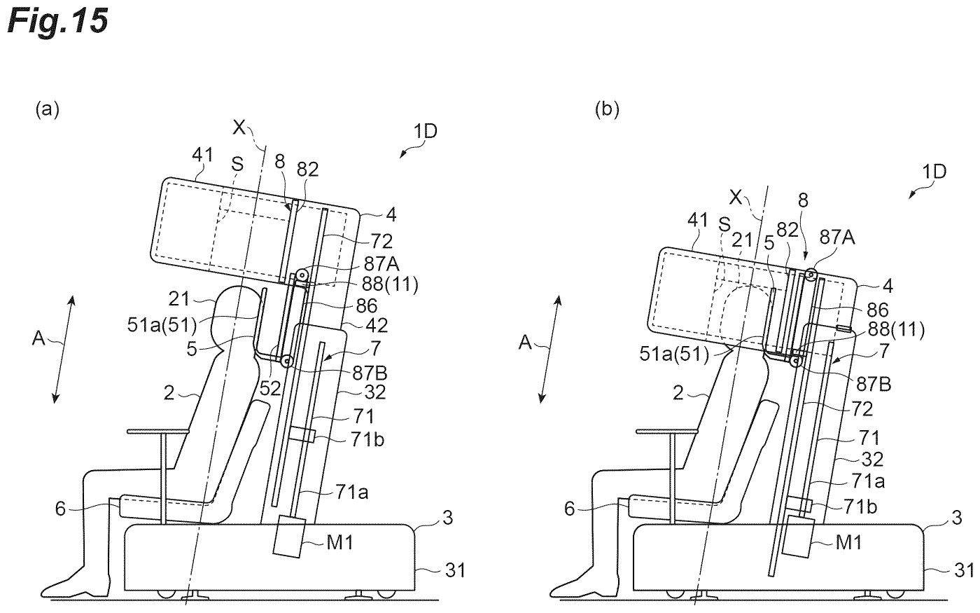

The clutch 88 functions as the switching unit 11 that switches the connection state between the first motor M1 and the second moving mechanism 8. The clutch 88 connected between the first motor M1 and one pulley 87A (the second moving mechanism 8) switches a connection state between the first motor M1 and the pulley 87A between a first state in which the first motor M1 and the pulley 87A are connected to each other and a second state in which the first motor M1 and the pulley 87A are disconnected from each other. In the first state, the driving force of the first motor M1 is transmitted to the pulley 87A, while in the second state, the driving force of the first motor M1 is not transmitted to the pulley 87A. The second moving mechanism 8 has a power transmission mechanism that branches the driving force of the first motor M1 and transmits a part of the driving force to the pulley 87A, but illustration of the power transmission mechanism is omitted in FIGS. 13 to 15. An aspect of movement of the information detection unit 4 and the headrest 5 in each of the first state and the second state is similar to that of the head image acquisition device 1C of the third modified example. FIGS. 14 and 15 are diagrams for describing an example of a head image acquisition method using the head image acquisition device 1D of the fourth modified example. The head image acquisition method of the fourth modified example is similar to the head image acquisition method of the third modified example described with reference to FIGS. 12 and 13.

The present disclosure is not limited to the above embodiments and modified examples. For example, materials and shapes of each structure are not limited to the materials and shapes described above, and various materials and shapes can be employed. The head image acquisition device 1 may be configured as a CT device. In this case, an output unit that outputs radiation may be arranged inside the main body 41, and radiation output from the output unit and transmitted through the head 21 of the subject 2 may be detected by the detection unit 43. That is, the detection unit 43 may detect radiation emitted from the head 21 or may detect radiation passing through the head 21.

When the headrest 5 is connected to the information detection unit 4, the first motor M1 and the second motor M2 may be arranged in the base 3. For example, in the above embodiment, the headrest 5 may be connected to the information detection unit 4. In this case, the second moving mechanism 8 may be provided from the base 3 to the information detection unit 4. The direction in which the information detection unit 4 moves relative to the base 3 and the direction in which the headrest 5 moves relative to the base 3 do not necessarily have to be parallel to each other. The direction A does not necessarily have to be inclined with respect to the vertical direction, and may be a direction parallel to the vertical direction. The head image acquisition device 1 does not need to include the chair 6. The head image acquisition device 1 may include an indicator (for example, a lamp) for displaying whether or not the headrest 5 is arranged at a predetermined position, an operating state of the switching unit 11, etc. Even though the above embodiments and modified examples have been described using examples in which the actuator is a motor, the actuator is not limited to a motor. For example, the actuator may use pneumatic pressure, hydraulic pressure, etc. as a power source.

REFERENCE SIGNS LIST

1, 1A, 1B, 1C, 1D: head image acquisition device, 2: subject, 21: head, 3: base, 4: information detection unit, 43: detection unit, 5: headrest, 6: chair, 7: first moving mechanism, 8: second moving mechanism, 10: controller, 11: switching unit, M1: first motor (first actuator), M2: second motor (second actuator).

1. A head image acquisition device for acquiring an image of a head of a subject in a sitting state, the head image acquisition device comprising:
a base;
an information detection unit including a detection unit that detects radiation from the subject; and
a headrest to which the head of the subject is fixed, wherein:
the information detection unit is connected to the base so that the information detection unit is movable relative to the base, and
the headrest is connected to one of the information detection unit and the base so that the headrest is movable relative to the one of the information detection unit and the base.

2. The head image acquisition device according to claim 1, wherein a direction in which the information detection unit moves relative to the base is parallel to a direction in which the headrest moves relative to the one of the information detection unit and the base.

3. The head image acquisition device according to claim 1, further comprising:
a first actuator for moving the information detection unit relative to the base; and
a second actuator for moving the headrest relative to the one of the information detection unit and the base.

4. The head image acquisition device according to claim 3, further comprising a controller that controls operations of the first actuator and the second actuator, wherein:
the one of the information detection unit and the base is the base, and
the controller performs movement control to control the first actuator so that the information detection unit approaches the base in a state in which the second actuator is stopped so that a distance between the information detection unit and the headrest decreases while a position of the headrest relative to the base is maintained at the same position.

5. The head image acquisition device according to claim 3, further comprising a controller that controls operations of the first actuator and the second actuator, wherein:
the one of the information detection unit and the base is the information detection unit, and
the controller performs movement control to control the second actuator so that the headrest approaches the information detection unit while the controller controls the first actuator so that the information detection unit approaches the base so that a distance between the information detection unit and the headrest decreases while a position of the headrest relative to the base is maintained at the same position.

6. The head image acquisition device according to claim 3, wherein the first actuator and the second actuator are arranged in the base.

7. The head image acquisition device according to claim 3, wherein:
the one of the information detection unit and the base is the information detection unit, and
the first actuator is arranged in the base, and the second actuator is arranged in the information detection unit.

8. The head image acquisition device according to claim 1, further comprising an actuator for moving the information detection unit relative to the base and moving the headrest relative to the one of the information detection unit and the base.

9. The head image acquisition device according to claim 8, further comprising:
a first moving mechanism that moves the information detection unit relative to the base by a driving force of the actuator;
a second moving mechanism that moves the headrest relative to the one of the information detection unit and the base by a driving force of the actuator; and
a switching unit that switches a connection state between the actuator and the second moving mechanism.

10. The head image acquisition device according to claim 9, wherein:
the one of the information detection unit and the base is the base,
in a state in which the actuator and the second moving mechanism are connected to each other, the information detection unit is movable relative to the base by the first moving mechanism, and the headrest is movable relative to the base by the second moving mechanism, and
in a state in which the actuator and the second moving mechanism are disconnected from each other, the information detection unit is able to approach the base by the first moving mechanism so that a distance between the information detection unit and the headrest decreases while a position of the headrest relative to the base is maintained at the same position.

11. The head image acquisition device according to claim 9, wherein:
the one of the information detection unit and the base is the information detection unit,
in a state in which the actuator and the second moving mechanism are disconnected from each other, the information detection unit is movable relative to the base by the first moving mechanism, and
in a state in which the actuator and the second moving mechanism are connected to each other, the headrest is able to approach the information detection unit by the second moving mechanism while the information detection unit approaches the base by the first moving mechanism so that a distance between the information detection unit and the headrest decreases while a position of the headrest relative to the base is maintained at the same position.

12. The head image acquisition device according to claim 1, further comprising a chair connected to the base for the subject to sit on.

13. A head image acquisition method using the head image acquisition device according to claim 1, the one of the information detection unit and the base being the base, the head image acquisition method comprising, in this order, steps of:
adjusting a position of the headrest by moving the headrest relative to the base;
fixing a head of the subject to the headrest; and
positioning the information detection unit and the headrest at positions where an image of the head is able to be acquired by the information detection unit by moving the information detection unit relative to the base.

14. A head image acquisition method using the head image acquisition device according to claim 1, the one of the information detection unit and the base being the information detection unit, the head image acquisition method comprising, in this order, steps of:
adjusting a position of the headrest by moving the information detection unit relative to the base;
fixing a head of the subject to the headrest; and
positioning the information detection unit and the headrest at positions where an image of the head is able to be acquired by the information detection unit by moving the headrest relative to the information detection unit while moving the information detection unit relative to the base.
